(19) United States
(12) Patent Application Publication
(10) Pub. No.: US 2006/0194243 A1
(43) Pub. Date: Aug. 31, 2006

(54) Methods for Identifying DNA Copy Number Changes

(75) Inventors: Rajeswari P. Tadakamalla, Sunnyvale, CA (US); Kai Wu, Mountain View, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, Inc., Santa Clara, CA (US)

(21) Appl. No.: 11/381,813

(22) Filed: May 5, 2006

Related U.S. Application Data
(62) Division of application No. 11/199,742, filed on Aug. 8, 2005.
(60) Provisional application No. 60/599,334, filed on Aug. 6, 2004.
(60) Provisional application No. 60/671,019, filed on Apr. 12, 2005.

(51) Int. Cl. C12Q 1/68; G06F 19/00

(52) U.S. Cl. 435/6; 702/20

(57) ABSTRACT

Methods and computer software products for identifying changes in genomic DNA copy number are disclosed. Methods for identifying homozygous deletions and genetic amplifications are disclosed. Genomic DNA is amplified generically and amplified sample is hybridized to an expression array. The expression array comprises probes to regions of genes that are expressed. The probes are complementary to genomic sequences found in mRNAs. Signal intensity is correlated to copy number. The methods may be used to detect copy number changes in cancerous tissue compared to normal tissue. The methods may be used to diagnose cancer and other diseases associated with chromosomal anomalies.

### PRIORITY

The present application claims priority to U.S. Provisional Application Nos. 60/599,334 filed Aug. 6, 2004 and 60/671,019 filed Apr. 12, 2005, the entire disclosures of which are incorporated herein by reference in their entireties for all purposes.

### FIELD OF THE INVENTION

The invention is related to methods of estimating the number of copies of one or more genomic regions that are present in a sample using oligonucleotide microarrays. Specifically, this invention provides methods, computer software products and systems for the detection of regions of chromosomal amplification and deletion from a biological sample.

### BACKGROUND OF THE INVENTION

The underlying progression of genetic events which transform a normal cell into a cancer cell is characterized by a shift from the diploid to anueploid state (Albertson et al. (2003), Nat Genet, Vol. 34, pp. 369-76 and Lengauer et al. (1998), Nature, Vol. 396, pp. 643-9). As a result of genomic instability, cancer cells accumulate both random and causal alterations at multiple levels from point mutations to whole-chromosome aberrations. DNA copy number changes include, but are not limited to, loss of heterozygosity (LOH) and homozygous deletions, which can result in the loss of tumor suppressor genes, and gene amplification events, which can result in cellular proto-oncogene activation. One of the continuing challenges to unraveling the complex karyotype of the tumor cell is the development of improved molecular methods that can globally catalogue LOH, gains, and losses with both high resolution and accuracy.

Numerous molecular approaches have been described to identify genome-wide LOH and copy number changes within tumors. Classical LOH studies designed to identify allelic loss using paired tumor and blood samples have made use of restriction fragment length polymorphisms (RFLP) and, more often, highly polymorphic microsatellite markers (STRS, VNTRs). The demonstration of Knudson's two-hit tumorigenesis model using LOH analysis of the retinoblastoma gene, Rb 1, showed that the mutant allele copy number can vary from one to three copies as the result of biologically distinct second-hit mechanisms (Cavenee, et al. (1983), Nature, Vol. 305, pp. 779-84.). Thus regions undergoing LOH do not necessarily contain DNA copy number changes.

Approaches to measure genome wide increases or decreases in DNA copy number include comparative genomic hybridization (CGH) (Kallioniemi, et al. (1992), Science, Vol. 258, pp. 818-21.), spectral karyotyping (SKY) (Schrock, et al. (1996), Science, Vol. 273, pp. 494-7.), fluorescence in situ hybridization (FISH) (Pinkel et al. (1988), Proc Natl Acad Sci USA, Vol. 85, pp. 9138-42), molecular subtraction methods such as RDA (Lisitsyn et al. (1995), Proc Natl Acad Sci USA, Vol. 92, pp. 151-5; Lucito et al. (1998), Proc Natl Acad Sci USA, Vol. 95, pp. 4487-92), and digital karyotyping (Wang, et al. (2002), Proc Natl Acad Sci USA, Vol. 99, pp. 16156-61). CGH, perhaps the most widely used approach, uses a mixture of DNA from normal and tumor cells that has been differentially labeled with fluorescent dyes. Target DNA is competitively hybridized to metaphase chromosomes or, in array CGH, to cDNA clones (Pollack et al. (2002), Proc Natl Acad Sci USA, Vol. 99, pp. 12963-8) or bacterial artificial chromosomes (BACS) and PI artificial chromosomes (PACs) (Snijders et al. (2001), Nat Genet, Vol. 29, pp. 263-4, Pinkel, et al. (1998), Nat Genet, Vol. 20, pp. 207-11). Hybridization to metaphase chromosomes, however, limits the resolution to 10-20 Mb, precluding the detection of small gains and losses. While the use of arrayed cDNA clones allows analysis of transcriptionally active regions of the genome, the hybridization kinetics may not be as uniform as when using large genomic clones. Currently, the availability of BAC clones spanning the genome limits the resolution of CGH to 1-2 Mb, but the recent use of oligonucleotides improves resolution to about 15 Kb (Lucito et al. (2003), Genome Res, 13:2291-305). CGH, however, is not well-suited to identify regions of the genome which have undergone LOH such that a single allele is present but there is no reduction in copy number.

### SUMMARY OF INVENTION

Methods for estimating copy number of selected genomic regions are disclosed. In a preferred embodiment genomic DNA is amplified by a whole genome amplification method such as multiple displacement amplification which uses a strand displacing polymerase and random primers to prime synthesis of cDNA.

The amplified genomic sample is labeled and hybridized to a high density array of probes. The array comprises more than 400,000, more than 700,000 or more than 1,000,000 different oligonucleotide probes. Each different probe is present in multiple copies in a discrete location or feature on the array. The location of each probe is known or determinable. In preferred embodiments the probes are between 15 and 60 bases and in a more preferred embodiment the probes are about 25 bases in length.

In a preferred embodiment the array is an expression array that comprises probe sets to detect mRNA transcripts from known genes. The array may contain probe sets to the expressed regions of more than 10,000, more than 30,000 or more than 40,000 genes. In preferred aspects the expression array is a human expression array.

In another embodiment computer implemented methods for analysis of hybridization data to estimate copy number are disclosed.

### DETAILED DESCRIPTION OF THE INVENTION

General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. Ser. Nos. 09/916,135, 09/920,491 (U.S. Patent Application Publication 20030096235), 09/910,292 (U.S. Patent Application Publication 20030082543), and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (U.S. Publication Number 20020183936), 10/065,856, 10/065,868, 10/328,818, 10/328,872, 10/423,403, and 60/482,389.

Definitions

The term “admixture” refers to the phenomenon of gene flow between populations resulting from migration. Admixture can create linkage disequilibrium (LD).

The term “allele” as used herein is any one of a number of alternative forms a given locus (position) on a chromosome. An allele may be used to indicate one form of a polymorphism, for example, a biallelic SNP may have possible alleles A and B. An allele may also be used to indicate a particular combination of alleles of two or more SNPs in a given gene or chromosomal segment. The frequency of an allele in a population is the number of times that specific allele appears divided by the total number of alleles of that locus.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a biopolymer is a “biomonomer”.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “genotype” as used herein refers to the genetic information an individual carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions.

The term “Hardy-Weinberg equilibrium” (HWE) as used herein refers to the principle that an allele that is homozygous leads to a disorder that prevents the individual from reproducing does not disappear from the population but remains present in a population in the undetectable heterozygous state at a constant allele frequency.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na⁺], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490 and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analog that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage analysis” as used herein refers to a method of genetic analysis in which data are collected from affected families, and regions of the genome are identified that co-segregated with the disease in many independent families or over many generations of an extended pedigree. A disease locus may be identified because it lies in a region of the genome that is shared by all affected members of a pedigree.

The term “linkage disequilibrium” or sometimes referred to as “allelic association” as used herein refers to the preferential association of a particular allele or genetic marker with a specific allele, or genetic marker at a nearby chromosomal location more frequently than expected by chance for any particular allele frequency in the population. For example, if locus X has alleles A and B, which occur equally frequently, and linked locus Y has alleles C and D, which occur equally frequently, one would expect the combination AC to occur with a frequency of 0.25. If AC occurs more frequently, then alleles A and C are in linkage disequilibrium. Linkage disequilibrium may result from natural selection of certain combination of alleles or because an allele has been introduced into a population too recently to have reached equilibrium with linked alleles. The genetic interval around a disease locus may be narrowed by detecting disequilibrium between nearby markers and the disease locus. For additional information on linkage disequilibrium see Ardlie et al, Nat. Rev. Gen. 3:299-309, 2002.

The term “lod score” or “LOD” is the log of the odds ratio of the probability of the data occurring under the specific hypothesis relative to the null hypothesis. LOD=log [probability assuming linkage/probability assuming no linkage].

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but includes other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptor is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended. A “Probe Target Pair” is formed when two macromolecules have combined through molecular recognition to form a complex.

Copy Number Analysis on Expression Arrays

Cancer is often caused by an increase or decrease in the expression of one or more genes. Tumor cells frequently show amplification or deletion of genes which can result in activation of oncogenes. Amplification of the region containing the gene results in an increase in the expression of the gene, resulting in an inappropriate activation of the gene. Methods useful for correlation of the increase in expression with the increase in genomic copy number are disclosed. The detection of these changes has direct relevance to cancer diagnosis and therapy.

The methods are related to methods and computer software for analyzing copy number using genotyping arrays as disclosed in U.S. Patent Publication Nos. 20050130217 and 20050064476 and U.S. Provisional Application Nos. 60/694,102 and 60/633,179, which are incorporated herein by reference for all purposes.

Methods for detection of differences in DNA copy number by hybridization of genomic DNA to expression arrays are disclosed. In a preferred embodiment an expression array comprises a plurality of probes or probe sets that are complementary to genomic regions that are predicted to be present in mRNA transcripts. The probes present on an expression array target expressed regions of the genome and generally do not detect intergenic regions or regions that are not present in the processed mRNA, for example, regions of constitutively spliced introns. In many aspects an array comprising probe sets for more than 38,500 human genes and more than 47,400 predicted transcripts may be used. In a preferred aspect the array used may be the Affymetrix U133 Plus 2.0 array. The U133 array includes more than 54,000 probe sets. For most targets there are 11 perfect match/mismatch probe pairs for each target and a total of more than 1,300,000 different oligonucleotide probe sequences. The signal intensity at each probe in a probe set is used to calculate a signal value for the probe set. The signal is a quantitative metric which represents the relative level of a given genomic region in the sample. The signal is calculated from a plurality of measurements of the intensity of fluorescence or chemiluminscence from individual features. A plurality of measurements from the probes of a probe set is used to calculate a signal for a probe set. The signal is a normalized measurement. In another aspect the probes of the array may be attached to beads or optical fibers.

Signal may be calculated using algorithms that are typically used for expression analysis, for example, the Signal Algorithm as described in Data Analysis Fundamentals, Available from Affymetrix, Inc. Signal is calculated using the One-step Tukey's Biweight estimate, yielding a robust weighted mean that is relatively insensitive to outliers. Each probe pair in a probe set has a potential vote in the Signal value. The vote is defined as an estimate of the real signal due to hybridization of the target. The mismatch intensity is used to estimate stray or background signal. The real signal is estimated by taking the log of the Perfect Match intensity after subtracting the stray signal estimate. The probe pair vote is weighted more strongly if this probe pair Signal value is closer to the median value for a probe set. Once the weight of each probe pair is determined, the mean of the weighted intensity values for a probe set is identified. This mean value is corrected back to linear scale and is output as Signal.

Unlike with expression analysis where transcripts are present at different levels, reflecting the amount of expression of individual genes, genomic regions should be present at approximately the same levels, unless a duplication or deletion has occurred. As a result it is expected that the Signal calculated for genomic regions should generally be similar. In the present methods, differences in the calculated Signal are used to indicate regions of the genome that have been amplified. In general, most probe sets should give approximately the same Signal. Those probe sets that show Signals that are much more than other probe sets may be identified as regions of amplification. The amount of amplification is proportional to the increase in Signal relative to the Signal of probe sets for regions that are not amplified.

In a preferred embodiment the tumor sample is amplified and hybridized to the array and estimations of copy number for genomic regions that may be expressed are made by comparison of hybridization patterns for probe sets in that region to hybridization patterns for probe sets in other regions. The comparisons may be made between probe sets on the same array in the same hybridization experiment instead of comparing hybridizations from separate arrays that may be from separate samples. Absent amplification or deletion most genomic regions will be present at the same level in a sample from a diploid organism so the Signal from a probe set for a first genomic region should be similar to the Signal from a probe set for all other genomic regions. The genomic regions targeted by the probe sets of the array preferably correspond to mRNAs. If a region is amplified the signal will increase over the signal of the majority of the probe sets. The increase is proportional to the amount of amplification, although it may not be a one to one correspondence.

Differences in chromosomal copy number have been detected by hybridizing fluorescently labeled DNA to metaphase chromosome spreads, arrays of BAC DNA (Gray et al.) and cDNA arrays (Pollack et al.). Methods for detecting changes in DNA copy number using arrays of probes that are complementary to expressed regions of genes are disclosed. The methods may be used to identify amplifications and deletions that alter coding regions, for example, to identify oncogenes such as Her2/Neu that are amplified in many cancers.

In a preferred embodiment, genomic DNA is amplified, the amplified DNA is fragmented and labeled and hybridized to an array of oligonucleotide probes targeting expressed regions of a genome. A small amount of genomic DNA can be used for amplification, in some aspects between 1 and 100 ng is sufficient and less than 1 ng may also be used. In a preferred embodiment the array is an expression array. The probes on an expression array are designed to detect mRNA targets, typically mRNAs are amplified and labeled and the labeled amplification products are detected. The design of the probes, for example, sense or antisense, will depend on the amplification method used. Typically mRNA is the region of the genome that is transcribed from genes, processed from pre-mRNA to mRNA and translated into proteins. The array of probes contains hundreds of thousands of probe sequences each present at a different known location on the array. Each feature of the array contains a different probe sequence and each probe sequence is complementary to a different region of the genome. Many copies of the same probe are present in each feature.

Expression analysis of tumors and copy number analysis may be performed using separate copies of the same array and in some embodiments expression and copy number may be measured simultaneously on a single array using two distinct labels. The amplified expression product is labeled with one label and the amplified genomic DNA is labeled with a second differentially detectable label. In another aspect, copy number and expression levels may be determined using duplicate copies of the same array and the samples may be labeled with the same label. Expression levels may be correlated with gene copy number.

In one embodiment genomic DNA may be amplified using a method that amplifies the genome in a relatively unbiased manner. One method of whole genome amplification (WGA) that may be used includes incubation of genomic DNA with random primers and a strand displacing polymerase, such as phi29 under isothermal conditions. This method has been described, for example, in U.S. Pat. Nos. 6,642,034 and 6,617,137 and in Dean et al. (2002) Proc. Natl. Acad. Sci. USA 99:5261-5266, Hosono et al. (2003) Genome Res. 13:954-964, Hosono et al. (2003) Genome Res. 13:954-964, and Yan et al. (2004) Biotechniques 37:136-143. Phi 29 variants have been described in, for example, U.S. Pat. No. 5,576,204. Whole genome amplification kits are available, for example, Molecular Staging Inc., makes a kit for Multiple Displacement Amplification (MDA) and the GenomePhi kit is available from Amersham Biosciences. Rubicon genomics also sells the GenomePlex whole genome amplification kit which may be used. The Rubicon methods are described, for example in, U.S. Patent Publication No. 20030143599. Multiple displacement amplification results in a relatively unbiased amplification of essentially all genomic regions and is particularly well suited for use with the present methods, see Paez, J. G. et al. Nucleic Acids Research 32(9), e71, 2004.

Genomic samples prepared by methods that result in a reduction in complexity may also be used for copy number analysis. The Whole Genome Sampling Assay (WGSA) in combination with genotyping arrays has been used for genotyping analysis (see for example Kennedy, G. C. et al. Nature Biotechnology 21, 1233-7, 2003, Matsuzaki, H. et al. Genome Research 14(3), 414-25, 2004 and Liu, W. et al. Bioinformatics 19, 2397-403, 2003) as well as for copy number analysis, (see Huang, J. et al. Human Genomics 1(4), 287-99, 2004, Bignell, G. R. et al. Genome Research 14(2), 287-95, 2004, Zhao, X. et al. Cancer Research 64(9), 3060-71, 2004). Other methods of copy number analysis using reduced complexity samples have also been reported (see, Lucito et al. (2003), Genome Res, 13:2291-2305 and Sebat et al. Science 305:525-528 (2004).

A probe set for a given transcript may comprise between 2 and 25 probe pairs. In some aspects probe sets are comprised of a plurality of probe pairs, a probe pair comprises a perfect match probe and a mismatch probe. The perfect match probes in a given probe set differ in the region of the gene that each probe is complementary to. In a preferred aspect most of the probe sets have 11 probe pairs. Probes may be complementary to overlapping or non-overlapping regions of a gene. For example, a first probe may be complementary to bases 200-224 and a second probe may be complementary to bases 210-234, these probes are overlapping. An example of non-overlapping probes would be a first probe complementary to bases 200-224 and a second probe complementary to bases 220-244. Probes may also be complementary to immediately adjacent regions, for example 200-224 and 225-249.

The signal value is calculated for a given probe set. Use of a plurality of probes in a probe set allows for a more accurate normalized measurement that is not as sensitive to the behavior of individual probes. Outlier probes can be thrown out of the calculation of signal.

Probes of a probe set may be designed to target specific regions of a transcript. For example, most of the probes in a probe set may be targeted to the 3′ end of an mRNA, for example the last 600 bases. Other arrays may target the final 300 bases of the mRNA. In another embodiment probes to each predicted exon of transcripts may be included. All exon arrays are described in U.S. patent application Ser. Nos. 11/036,498 and 11/036,317. All references cited above are incorporated herein by reference in their entireties for all purposes.

In a preferred aspect the data is analyzed using four assumptions. First, the majority of probe sets have normal copy number. Second, the hybridization behavior of probe sets follows a normal distribution. Third, the deletion and amplification occurs at variable locations within individual DNA samples. Fourth, the DNA copy number reflected in probe set signal is a signal of strength of the analysis.

In a preferred aspect there are six steps to data processing of probe sets. The first is normalization of the data points and this aspect uses the trimmed mean approach. The probe set signal on a chip is scaled back to 250. The data is sorted and 2.5% of all data at either extreme is trimmed for each array. The remaining data in the middle is used to compute mean for each array. The scaling factor for each array is determined by comparing trimmed mean approach with 250. For each array, all probe set signal is scaled by this factor. In initial embodiments probe sets that were determined to be well behaving or “good” probe sets on chromosomes 21 and 22 were used for scaling and a statistical algorithm using a trimmed-mean approach for scaling Chr21 and Chr22 reference probe sets to 250 was used. In another embodiment, all probe sets on the array are used for global scaling. This is beneficial because it takes into consideration that Chr21 and Chr22 may have deletions or amplifications and that the majority of genes are not expected to be amplified or deleted.

The second step of data processing is data partitioning into training and test sets. In order to ascertain copy number change, a standard for comparison is used. Because an ideal reference set is not available a robust algorithm is used to handle intrinsic biases within data set. For this purpose, all data originating from different categories, different hybridization time, and different sources is combined. All data is partitioned into a relatively balanced training set and a test set. For example, four data categories may be used: Cell lines with 5X, 4X, 3X, 2X, 1X (no Y) chromosomes: 5, Cell lines with known deletions (e.g. Chr 13, 4, 8 and X): 4, Blood DNA from normal people (XX, XY): 10, and Human GIST tumor samples: 5. The criteria for selecting a training data set may be a mean of about 250 with a standard deviation range of 280 to 450. Experiments with very large standard deviations may be removed from the training set.

The third step in the data processing is generating a signal mean and a standard deviation from the training set.

The fourth step in the data processing is generating a Z-score for every probe set. The Z-score measures the distance of each sample from a reference mean. A Z-score is computed for each data point for each experiment by the following equation Z=(Xi-Meani)/SDi.

The fifth step in the data processing is obtaining probe sets (with Z-scores) mapped to chromosomal locations. Probe set locations with Z-scores are mapped with a parsed NetAffx annotation file. Some probe sets may map to multiple chromosomes and in a preferred embodiment those probe sets are removed from the analysis. When multiple probe sets clustered together on the same chromosome and show the same pattern of amplification or deletion, this adds statistical significance to the copy number estimate. A sliding window with combined Stouffer Z score may be used to graphically display the change. The Y-axis may be used to represent the position on the chromosome and the X-axis the signal intensity at the probe set or the Z-score. In preferred embodiments the data is transformed to a log2 scale.

The sixth step in the data processing is generating a Stouffer Z-score (F. M. Mosteller, and R. R. Bush, Selected quantitative techniques, In: G. Lindzey (ed.), Handbook of Social Psychology: Vol. 1. Theory and Method, Addison-Wesley, 1954, 289-334). The Stouffer Z-Score allows detection of copy number change within a user-defined sliding chromosomal window. A sliding window with combined Stouffer Z-score can graphically display copy number change. The new Stouffer Z-score represents the composite deviation of the mean in a window size of interest and is shown by the following equation: Zs=ΣZn/√n. The end user can set a value above or below a certain threshold of the Stouffer Z score that a deletion or amplification occurs.

Computerized methods and computer software products for analyzing hybridization data to expression arrays to estimate copy number are disclosed. The data analysis methods described are typically performed by computers. In some embodiments, a computerized method for analyzing hybridization data and analyzing copy number along a chromosome or region of a chromosome includes the steps of inputting probe intensities from multiple probes and obtaining a normalized signal intensity for each of a plurality of probe sets. The normalized signal intensities for a probe set are compared with neighboring probe sets (corresponding to contiguous genomic regions) and to probe sets from other regions of the genome. Changes in signal intensity are correlated with changes in the copy number of a genomic region. The boundaries of an amplified or deleted region may be estimated by looking at probe sets that detect contiguous genomic regions. In some aspects changes in copy number are correlated with changes in expression level by comparing copy number analysis to gene expression analysis at the probe set level (copy number analysis from a probe set can be compared to expression analysis using the same probes).

In one aspect of the invention, computer software products and computer systems are provided to perform the methods and algorithms described above. Computer software products of the invention typically include a computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Computer systems of the invention typically include at least one CPU coupled to a memory. The systems are configured to store and/or execute the computerized methods described above. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

### EXAMPLE

Single Label Array Copy Number Assay Protocol

The following reagents and materials were used. DNA samples containing various X-chromosome copy numbers (NA01723, NA09899, NA04626, NA01416 and NA06061) were acquired from Coriell Cell Repositories (Camden, N.J.). Reagents were as follows: REPLI-g™ obtained from Molecular Staging Inc (New Haven, Conn.); Qiagen Genomic-Tip 100 G (P/N 10243) and 500 G (P/N 10262); Qiagen Genomic DNA Buffer Set (P/N 19060); 10× Fragmentation Buffer (Affymetrix P/N 900422); GeneChip Fragmentation Reagent (Affymetrix P/N 900131); GeneChip DNA Labeling Reagent (Affymetrix P/N 900484); Terminal Deoxynucleotidyl Transferase (Affymetrix P/N 900426); TdT Buffer (Affymetrix P/N 900425): 4% TBE Gel, BMA Reliant precast (4% NuSieve 3:1 Plus Agarose); (CAMBREX, P/N 54929): YM-3 Microcon column (Millipore, P/N 42404); MES Free Acid Monohydrate (Sigma-Aldrich, P/N M5287); MES Sodium Salt (Sigma-Aldrich, P/N M5057); (12× MES solution: dissolve 70.4 g MES free acid monohydrate and 193.3 MES sodium salt in 800 ml Molecular Biology Grade water, adjust volume to 1 Liter and filter through a 0.2 μM filter); 5M TMACL (Tetramethyl Ammonium Chloride); (Sigma P/N T3411); 1% Tween-20: Pierce; Catalog#: CAS9005-64-5; DMSO (Sigma P/N D5879); 0.5 M EDTA (Ambion, P/N 9260G); 50× Denhardts (Sigma; P/N D2532); Human Cot-1 (Invitrogen, P/N 15279-011); Oligo B2 (Affymetrix, P/N 500702 B2); 20×SSPE (Accugene, P/N 16-010); 10% Tween-20 (Pierce, P/N 28320); 5M TMACL (Tetramethyl Ammonium Chloride); (Sigma P/N T3411); 1% Tween-20: Pierce; Catalog#: CAS9005-64-5; 12×MES solution (see Technical Manual for Expression); DMSO (Sigma P/N D5879); Molecular Biology Grade water (Accugene, P/N 51200); ImmunoPure Streptavidin (Pierce; P/N: 21122); Acetylated BSA (Invitrogen); SAPE (Streptavidin, R-phycoerythrin conjugate) (Molecular Probes, P/N S866); Biotinylated Anti-Streptavidin (Vector; P/N: BA-0500, 0.5 mg/mL); Goat IgG (Sigma-Aldrich, P/N 15256). The array used was HU-133A Plus 2.0 (Affymetrix, P/N 900466).

Whole Genome Amplification. The DNA to be amplified is first denatured. The REPLI-g kit from Molecular Staging is used according to the procedure recommended by the manufacture. Briefly, 10-25 ng genomic DNA in 2.5 μl is denatured by adding 2.5 μl of freshly prepared Denaturation Solution from the kit, mixing and allowing the mixture to sit at room temp for 3 min. Then 5 μl of freshly prepared Denaturation Solution is added.

The denatured DNA is then amplified. For each 100 μl reaction, prepare the following reaction mixture: 10 μl of denatured genomic DNA, 25 μl of 4× reaction mix, 1 μl of DNA Polymerase, and 64 μl of distilled water. The reaction mixture is mixed well, transferred to an incubator or thermo cycle controller at 30° C. and incubated for 16 hours. The reaction is stopped by incubation at 65° C. for 10 minutes and then held at 4° C. Then the amplification product is purified using the Qiagen genomic-tip kit as described in the manufacturer's handbook, using a swinging bucket rotor. Briefly, for each 100 μl reaction, add 4.9 ml of QBT buffer ready to be applied to an equilibrated genomic-tip 100 column. For multiple reactions add QBT up to 20 ml buffer ready to be applied to an equilibrated genomic-tip 500 column. The DNA pellets are resuspended in 0.1-0.5 ml of distilled water so the final concentration of DNA is approximately 1.5 μg/μl and the DNA is measured using an OD260.

The next step is fragmentation of the amplification product. First, make a dilution of DNase I for a final concentration of 0.125 U/μl. To make the dilution mix 4.8 μl of 10× fragmentation buffer, 2 μl of DNase I (3.0 U/μl), and 41.2 μl of distilled water. Prepare the fragmentation reaction mix by mixing 100 μg of amplification product (up to 88 μl), 10 μl of 10× fragmentation buffer, 2 μl of DNase I (0.10 U/μl) and distilled water up to a final volume of 100 μl. The reaction is incubated at 37° C. for 30 minutes and then the reaction is stopped by incubation at 95° C. for 10 minutes. Then the reaction is cooled at 4° C. The reaction mix may be stored at −20° C. if not proceeding immediately to the labeling reaction. The fragmentation reaction may be analyzed for completeness by running 1 μl on a 4% NuSieve (3:1) pre-cast agarose gel along with 25 and 100 base pair marker ladders. The fragmentation should be a smear with the majority of the intensity between 25 and 200 base pairs.

The next step is labeling of the fragmented product. The labeling reaction is prepared as follows: mix 99 μl of the fragmentation product, 30 μl of 5×TdT Buffer, 13 μl of TdT (30 U/μl), and 8 μl of DNA Labeling Reagent (7.5 mM). The reaction is incubated at 37° C. for 5 hours. The incubation is stopped by incubation for 10 minutes at 95° C. and then cooled at 4° C. If not going immediately to the next step, the reaction mix can be stored at −20° C. The labeled product is cleaned using a YM-3 Microcon column. For detailed procedure see the label inserts by the manufacturer, but they are generally as follows: 1) Add 100-300 μg of labeled product to the column and spin at top speed of a microcentrifuge for 30 min 2) Add 300 μl of distilled water to the column and spin at top speed of a microcentrifuge for 30 min 3) Reverse the column and spin at 3,000 rpm for 5 min to collect the sample. The final concentration should be greater than 2 μg/μl. Store at −20° C. if necessary.

The next step is hybridization of the labeled product to an array of probes. The hybridization mix is prepared as follows: 100 μg (up to 41 μl) of labeled fragments, 100 μl of 2× Hybridization Mix, 25 μl of Human Cot-1 (1 μg/μl), 10 μl of 50× Denhardts Solution, 20 μl of 100% DMSO, 4 μl of 3 nM oligo B2, and distilled water up to a final volume of 200 μl. The hybridization mix is heated at 95° C. and then immediately cooled at 4° C. Then 200 μl of hybridization mix is added to the HLU-133A Plus 2.0 Array and hybridized at 48° C. for 16 hours at 60 rpm. The concentration of Denhardts solution in this hybridization has been increased from 1× to 2.5× and the concentration of human cot-1 DNA has been reduced to 25 μg from 50 μg.

The next step is the pre-washing of the hybridized product. The TMAC1 wash buffer is prepared in the following way: 1 ml of 20× SSPE, 25 ml of 5 M TMAC1, 0.05 ml of 10% Tween 20, and 23.95 ml of distilled water. Remove the hybridization mix from the array and fill the array with 200 μl TMAC1 wash buffer. Incubate the array in the hybridization oven for 30 minutes at 50° C. at 60 rpm.

The next step is washing and staining the array. Before these steps can occur, a series of solutions must be prepared first. The first solution is Wash Buffer A which is a low-stringent buffer (6×SSPE with 0.01% Tween 20). Wash Buffer A is prepared as follows: mix 300 ml of 20×SSPE, 1 ml 10% Tween 20, and 699 ml of distilled water and filter through a 0.2 μM filter. The second solution is Wash Buffer B which is a high stringent buffer (0.6×SSPE with 0.01% of Tween 20). Previously 0.3×SSPE was used in the high stringent buffer. Wash Buffer B is prepared as follows: mix 30 ml of 20×SSPE, 1 ml of 10% Tween 20, and 984 ml of distilled water and filter through a 0.2 μM filter. The third solution is a 2× Staining Buffer and it is prepared as follows: mix 41.7 ml of 12×MES, 92.5 ml of 5M NaCl, 2.5 ml 10% Tween 20, and 113.3 ml of distilled water. The fourth solution is the Streptavidin solution and it is prepared as follows: mix 300 μl of 2× Staining Buffer, 24 μl of 50 mg/ml acetylated BSA, 6 μl of 1 mg/ml Streptavidin, and 270 μl of distilled water. The fifth solution is the antibody solution and it is prepared as follows: mix 300 μl of 2× Staining Buffer, 24 μl of 50 mg/ml acetylated BSA, 6 μl of 10 mg/ml Goat IgG, 6 μl of 0.5 mg/ml Biotinylated Antibody, and 264 μl of distilled water. The sixth solution is the SAPE solution and it is prepared as follows: mix 300 μl of 2× Staining Buffer, 24 μl of 50 mg/ml acetylated BSA, 6 μl of 1 mg/ml SAPE, and 270 μl of distilled water.

First, the probe array is washed using the Fluidics Station 450 as follows: Post Hybe Wash 1: 10 cycles of 5 mixes/cycle, with Wash Buffer A at 35° C. Post Hybe Wash 2: 40 cycles of 10 mixes/cycle with Wash Buffer B at 50° C. Stain: 10 minutes in the Streptavidin Solution Mix at 35° C. Post Stain Wash: 10 cycles of 4 mixes/cycle with Wash Buffer A at 35° C. Second Stain: 10 minutes in the Antibody Solution Mix at 35° C. Third Stain: 10 minutes in SAPE Solution at 35° C. Finally, the probe array is washed for 15 cycles of 4 mixes/cycle with Wash Buffer A at 35° C. The holding temperature is 25° C.

The probe array is then scanned and analyzed as specified in the HU133A Plus 2.0 array inserts. In general the percent present calls are higher than about 70% and this may be used as a cutoff so that samples that have less than 70% present calls are determined to have failed.

Data were analyzed with the Affymetrix MAS 5.0 algorithm, and normalized to 250 using the global normalization approach. Data were partitioned into training set (37) and test set (23). Signal mean and Standard deviation (S.D.) were generated from the training set. Based on Signal and S.D. a Z score (copy number estimate) was generated for every probe set, which measures distance of each sample from reference mean. Z score calculation: for each probe set, compute Zi=(Xi-u)/sigma. Here Xi is the measured sample signal; u is the mean of the reference set and sigma is the standard deviation of the reference set. Probe sets (with Z score) were mapped to chromosomal locations. The results may be displayed using Microsoft Excel and Affymetrix Intergrated Genome Brower.

Stouffer-Z score calculation: take a window of 270 Kb, 135 kb upstream and 135 kb downstream for each probe set, then calculate sum (Zi)/square root (N). Sum (Zi) is the summation of all Z scores that fall within the 270 kb windows. N is the number of probe sets within the 270 kb window. The purpose of Stouffer Z is to calculate the “neighboring effect” of many probe set clustered in adjacent chromosome location, so that when adjacent probe sets all have positive (amplification) or negative (deletion) Z scores, the additive effect is significant. The benefit is that concordant changes around a chromosomal region are significantly amplified, whereas the effect of a few outliers is reduced. The outlier reduction relies on the number of data points within a window (sliding window size).

### CONCLUSION

Methods of identifying changes in genomic DNA copy number are disclosed. Methods for identifying loss of heterozygosity, homozygous deletions and gene amplifications are disclosed. The methods may be used to detect copy number changes in cancerous tissue compared to normal tissue. A method to identify genome wide copy number gains and losses by hybridization to an expression array comprising probes for more than 30,000 human transcripts is disclosed. Copy number estimations across the genome are linked to intensity of (LOH analysis). All cited references are incorporated herein by reference for all purposes.

The present inventions provide methods and computer software products for estimating copy number in genomic samples. It is to be understood that the above description is intended to be illustrative and not restrictive. Many variations of the invention will be apparent to those of skill in the art upon reviewing the above description. By way of example, the invention has been described primarily with reference to the use of a high density oligonucleotide array, but it will be readily recognized by those of skill in the art that other nucleic acid arrays, other methods of measuring signal intensity resulting from genomic DNA could be used. The scope of the invention should, therefore, be determined not with reference to the above description, but should instead be determined with reference to the appended claims, along with the full scope of equivalents to which such claims are entitled.

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

1-11. (canceled)

12. A method for identifying chromosomal regions of amplification or deletion comprising:
amplifying the genomic sample to obtain an amplified genomic sample;
fragmenting the amplified genomic sample to obtain fragments;
labeling the fragments;
hybridizing the fragments to an expression array to generate a hybridization pattern;
analyzing the hybridization pattern to obtain a plurality of probe set signals, wherein a probe set signal is a normalized measurement of the hybridization signal for a probe set;
calculating a Z-score for each probe set using a mean and standard deviation calculated from a training data set;
mapping the chromosomal location of each probe set to obtain a plurality of mapped probe sets that map to a single chromosomal location;
calculating a Stouffer Z-score for each mapped probe set; and
identifying chromosomal regions of amplification or deletion based on Stouffer Z-score.

13. The method of claim 12 wherein the genomic sample is amplified in a reaction comprising random primers and a strand displacing polymerase.

14. The method of claim 13 wherein the strand displacing polymerase is a phi29 DNA polymerase.

15. The method of claim 12 wherein the fragments are end labeled with biotin in a reaction comprising terminal transferase.

16. The method of claim 12 wherein the training data set is obtained by analyzing the probe set signal from at least 30 control genomic samples.

17. The method of claim 12 wherein the genomic samples included in the training data set each have a mean normalized probe set signal of about 250 and a standard deviation of between 280 and 450.

18. The method of claim 16 wherein the control genomic samples are normal samples.

19. The method of claim 12 wherein probe sets with a Stouffer Z-score above a selected threshold are identified as being complementary to an amplified genomic region.

20. The method of claim 12 wherein probe sets with a Stouffer Z-score below a selected lower threshold are identified as being complementary to a deleted genomic region.

21. A computer software product for analyzing hybridization data for a genomic sample hybridized to an expression array, comprising a computer readable medium having computer-executable instructions for performing logic steps comprising:
inputting probe intensities from probes designed to interrogate for the presence of mRNA transcripts;
obtaining a normalized signal for a plurality of probe sets;
partitioning the data into a training set and a test set;
generating a signal mean and a standard deviation from the training set;
generating a Z-score for a plurality of probe sets;
identifying probe sets that map to chromosomal locations and comparing Z-scores for probe sets to estimate copy number for selected genomic regions.

22. The computer software product of claim 21 wherein Stouffer Z-scores are obtained for a plurality of the probes sets and wherein the Stouffer Z-scores are plotted against chromosomal location and output as a graphical display.

23. The method of claim 13 wherein the strand displacing polymerase is a Bst DNA polymerase.

24. The method of claim 12 wherein the expression array comprises at least 10,000 probe sets.

25. The method of claim 24 wherein each probe set comprises a plurality of probe pairs, wherein each probe pair contains a perfect match probe and a mismatch probe.

26. The method of claim 12 wherein the probe set signals are determined using a plurality of probes in a probe set.

27. The method of claim 12 wherein the probe set signals are a mean of weighted intensity values, wherein the weighted intensity values are determined using a weight of each probe pair.

28. The method of claim 27 wherein each probe pair is weighted more strongly if a probe pair signal value is closer to a median value for the probe set.

29. The method of claim 12 wherein the normalized measurement of the hybridization signal is determined using a stray signal and a real signal, wherein the stray signal is estimated using mismatch intensity and the real signal is estimated by taking a log of a perfect match intensity after subtracting the estimated stray signal.
